(19) United States
(12) Patent Application Publication
(10) Pub. No.: US 2002/0138134 A1
(43) Pub. Date: Sep. 26, 2002

(54) Thermostent for biomedical use

(76) Inventors: Young Kon Kim, Busan (KR); Lee Hyun Shin, Busan (KR); Byeong Ho Park, Busan (KR)

Correspondence Address: Christopher W. Quinn, Brooks & Kushman P.C., 22nd Floor, 1000 Town Center, Southfield, MI, 48075-1351, US

(21) Appl. No.: 10/034,742

(22) Filed: Dec. 28, 2001

(30) Foreign Application Priority Data
Dec. 29, 2000 (KR) 2000-0085231
Dec. 29, 2000 (KR) 2000-0085233
Dec. 29, 2000 (KR) 2000-0085234

(51) Int. Cl. A61F 2/06

(52) U.S. Cl. 623/1.19; 606/158

(57) ABSTRACT

Disclosed are implants for use in biomedical treatment thermally, including thermo-coil, thermo-guide wire and thermostent. Thermally heated, magnetic material selected from among duplex stainless steel, nickel-copper alloy, iron-nickel alloy, palladium-cobalt alloy, and palladium-nickel alloy is fabricated into coils or tubular forms which can be inserted into the lumen. The material is treated at 200-1,500° C. After being inserted into the body, the implants can generate heat by themselves in response to the application of an external magnetic field, without a separate electrical connection to the exterior, thereby inducing necrosis or physiological changes at the target site and neighboring tissues to improve therapeutic effects at the target site. In addition to the hyperthermic effects, prevention of lumen restenosis and expansion from thermostent, interruption of blood flow can be obtained from the thermo coil, necrotization of unwanted tissue and closing of unwanted lumens from the thermo-guide wire.

### BACKGROUND OF THE INVENTION

1 Field of the Invention

The present invention relates, in general, to implants for use in the lumens of the body and, more particularly, to thermostent, thermoguide wire and thermocoil which can emit heat by themselves in the presence of magnetic fields and be inserted into lumens of the body to perform hyperthermia at body sites of interest, as well as functioning to maintain intraluminal passageway open and prevent the restenosis or expansion of the lumen.

2. Background Art

For some of patients who suffer from vascular diseases such as aneurysm, or from tumors, surgical operations cannot be operated. For example, patients suffering from vascular diseases as well as hypertension or heart disease cannot undergo a surgical operation. Surgical operations also cannot be performed on patients with tumors when patients are expected to be bled profusely.

Functioning as conduits for fluids such as blood or lymphs, a large number of lumens exist in the body. The dimensions of lumens may be altered, i.e., increased or decreased by diseases or adult disorders or certain other reasons. In either case, serious problems arise. For example, when the lumen becomes narrowed or obstructed, it cannot function well or at all. On the other hand, when the lumen is expanded, its wall is thinned to rupture. In these circumstances, therefore, the lumens in the body are required to retain their dimensions by artificial means to prevent restenosis or expansion. For use in such purposes, medical devices, called stents, were developed.

Assuming tubular forms for their functional purposes as a rule, stents are inserted into lumens to support passageways of lumens and to prevent restenosis or expansion of lumens.

Usually, stents are in the form of mesh type tubes, as disclosed in Korean Pat. Laid-Open Publication No. 1999-13858 and Korean Pat. No. 10-240832. However, the stents disclosed in the references may function only temporarily after being inserted. That is, such mesh type tubes are liable to be clogged because intraluminal tissues and cells of the progressive diseases migrate to inside of the stent and grow therein. Also, Korean Pat. Laid-Open Publication No. 2000-16119 refers to a tubular stent for angio, having different materials of the center portion and circumferential portion in a widthwise cross-section. This patent is also disadvantageous in light of intraluminal growth of cells.

U.S. Pat. No. 6,077,298 discloses a stent formed of a shape memory alloy with deformation temperature of 43-90° C., with connection through a conductive wire to an external power supply with the aid of which the stent is expanded and retracted. However, such accompaniments make it difficult to insert the stent and cause inconvenience the patients.

For medical use in such a case, thermocoils were developed. These coils are inserted into vessels around inflamed lesions to interrupt the blood flow flowing into the lesions to block the provision of nutrients thereto, thereby the lesions being cured, as disclosed in Korean Pat. Laid-Open Publication No. 1999-459.

With the aim of treating diseases, the conventional metal coil of the reference is inserted into blood vessels to block blood from flowing into a target. In the case of tumors, however, they cannot be fundamentally treated with the device that only interrupts blood flow.

Guide wire is used to safely introduce catheters into blood vessels of, for example, the heart. Various forms of guide wires are developed. Some of them are found in Japanese Pat. Publication Nos. Hei. 4-25024 and 7-10280 and Laid-Open Publication Nos. Hei. 2-4390 and 5-92044 and Korean Pat. No. 10-188237.

The conventional guide wires have various structures to easily apply catheters to the body. However, the guide wires are utilized only as subsidiaries to help catheters perform their therapeutic functions. Thus, the guide wire does not directly take part in the therapeutic treatment of diseases and must be removed after it guides the introduction of catheters into the body.

### SUMMARY OF THE INVENTION

Therefore, it is an object of the present invention to overcome the above problems encountered in prior arts and to provide thermo implants for use in hyperthermia.

It is another object of the present invention to provide a thermostent which can be inserted into lumens to maintain intraluminal passageways open and to prevent the intraluminal growth of tissues and the restenosis or expansion of the lumens and which generates heat by itself in the presence of an external magnetic field without a separate electrical connection, thereby inducing necrosis or physiological changes at the target site and neighboring tissues to improve therapeutic effects at the target site.

It is still a further object of the present invention to provide a thermocoil which can be inserted into blood vessels to interrupt the blood flow and can generate heat by itself in the presence of an external magnetic field without a separate electrical connection to the exterior, to maintain the target site at a predetermined temperature, whereby the target site and neighboring tissues are caused to undergo necrosis or physiological changes to improve therapeutic effects on the target site.

It is a further object of the present invention to provide a thermo-guide wire which can be inserted into lumens of the body to facilitate the safe and easy insertion of catheters and generate heat by itself in the presence of an external magnetic field without a separate electrical connection to the exterior, so as to maintain the target site at a predetermined temperature, thereby inducing necrosis or physiological changes at the target site and neighboring tissues to improve therapeutic effects at the target site.

In an aspect of the present invention, there is provided a thermostent for insertion into the lumen, having a mesh tubular form made of a heat-treated, magnetic material and generating heat by itself in response to the application of an external magnetic field.

In accordance with one aspect of the present invention, there is provided a thermocoil for insertion into the lumen, having a spiral form made of thermally treated, magnetic wire material and functioning to generate heat by itself in response to the application of an external magnetic field thereto and to block blood flow when being inserted into blood vessels.

In accordance with another aspect of the present invention, there is provided a thermoguide for insertion into the lumen, having a coil form made of thermally treated, magnetic wire material and generating heat by itself in response to the application of an external magnetic field.

A further aspect of the invention provides a method of generating heat within the lumen of a body which includes the insertion of a thermostent into the lumen and applying an external magnetic field to the thermostent to generate heat therein.

Yet another aspect of the invention provides a method of easing insertion of catheters into the lumen of a body by inserting a thermoguide wire into the lumen to facilitate the safe and easy insertion of catheters and wherein the thermoguide wire generates heat when an external magnetic field is applied.

The above objects and other objects, features, and advantages of the present invention are readily apparent from the following detailed description of the best mode for carrying out the invention when taken in connection with the accompanying drawings.

### BRIEF DESCRIPTION OF THE DRAWINGS

FIG. 1 is a diagram showing a spiral thermocoil in accordance with an embodiment of the present invention;

FIG. 2 is a schematic diagram showing a heat value-measuring apparatus;

FIG. 3 is a graph in which the temperature properties of the coil are plotted versus time;

FIG. 4 is a graph in which the heating rate of the coil is plotted versus the diameter;

FIG. 5 is a graph showing change of a magnetic permeability depending on nickel contents in iron-nickel alloy;

FIG. 6 is a graph in which heat values per unit weight and unit time of the duplex stainless steel wire with a diameter of 0.16 mm are plotted versus the heat treatment temperature;

FIG. 7 is a graph in which the heat values of the thermally treated duplex stainless steel wires used in the present invention are plotted versus the temperature of the wires;

FIG. 8 is a graph showing temperature gradient varying with distances from the stent in a pig liver;

FIG. 9 is a photograph showing protein denaturation of pig liver by a thermoguide wire encapsulated with a tube in accordance with the present invention;

FIG. 10 is a photograph showing protein denaturation of pig liver by a naked thermoguide wire in accordance with the present invention;

FIG. 11 is a graph showing heat value per unit weight and unit time, depending on heat treatment temperature;

FIG. 12 is a graph showing heating properties of the stent using the apparatus of FIG. 1;

FIG. 13 is a graph showing the heating properties of stents;

FIG. 14 is a graph showing the temperature distribution of the heat generated by stents in pig liver;

FIG. 15 is a photograph showing protein denaturation of pig liver by a general type 016 stent;

FIG. 16 is a photograph showing protein denaturation of pig liver by a general type 022 stent;

FIG. 17 is a photograph showing protein denaturation of pig liver by a wall type 016 stent; and

FIG. 18 is a photograph showing protein denaturation of pig liver by a wall type 022 stent.

### DETAILED DESCRIPTION OF THE PREFERRED EMBODIMENTS

The present invention is directed to medical implants which can generate heat by themselves when being subjected to an external magnetic field. In this regard, the implants of the present invention are made of thermally treated, magnetic materials.

Preferably, the magnetic material is selected from the group consisting of duplex stainless steel, nickel-copper alloy, iron-nickel alloy, palladium-cobalt alloy and palladium-nickel alloy.

To generate heat as high as 30-200° C., the material is thermally treated at 200-1,500° C.

Implants made of such magnetic materials are able to generate heat by themselves only by the application of an external magnetic field thereto, without any external electrical connection. In accordance with the present invention, the implants can be used in hyperthermia and include thermo-coils, thermo-guide wires, and thermo-stents.

Therefore, in one aspect, the present invention pertains to a thermostent which can be inserted into lumens, such as blood vessels, urinary tracts, bile ducts, gastrointestinal tracts, lymphatic ducts, and living tissues, to maintain intraluminal passageways open and to prevent the intraluminal growth of lumen endothelial cells and the restenosis or expansion of the lumens; and generates heat by itself in the presence of an external magnetic field without a separate electrical connection, whereby the target site and neighboring tissues are subjected to necrosis or physiological changes to improve therapeutic effects at the target site.

Also, in another aspect, the present invention pertains to a thermocoil which can be inserted into blood vessels to block blood flowing therein, as well as generating heat by itself under an external magnetic field to maintain the target site at a predetermined temperature, thereby causing necrosis or physiological changes at the target site and neighboring tissues to improve therapeutic effects of the target site.

Also, in a further aspect, the present invention pertains to a thermoguide wire which aids insertion of catheters into the lumens of patients with safety and ease, and which functions to generate heat by itself under an external magnetic field, thereby the heat necrotizing or physiologically changing the target site and neighboring tissues to improve therapeutic effects on the target site.

In order to better understand the present invention, a theoretical background for generating heat from a magnetic material is considered first.

Heat generation from the magnetic material according to the present invention is largely classified into two cases; first, the heat generated from eddy current loss caused by eddy current, that is, vortex current, under a magnetic field; and second, the heat attributed to hysteresis core loss created from a magnetic circuit formed in the magnetic material.

In general, when a conductor-penetrating magnetic flux is changed or a magnetic flux in a conductor changes with time owing to a relative motion between the magnetic flux and the conductor, a current is induced along a critical closed circuit formed locally in the conductor in order to prevent the flux change. Such current is referred to as an eddy current, which affects normal current distribution. At the same time, Joule heat is generated by the eddy current, thus inducing loss of electric power, a so-called eddy current loss.

When the magnetic field with a magnetic density of B=sinωt is applied in an axial direction of a cylinder having a radius ‘a’, a length ‘l’, a volume ‘V’ (=πa²l) and a resistance rate ‘ρ’, the magnetic flux ‘φ’ that penetrates a cross area of a radius ‘r’ (<a) has a relationship of φ=πr²Bₘsinωt, so that an electromotive force generated in a circumferential direction has the following equation:

$$e = - \frac{ⅆ φ}{ⅆ t} = - π {r}^{2} ϖ {B}_{m} cos ϖ t$$

In a cylinder with a fine thickness ‘dr’ by a radius ‘r’, a resistance versus the eddy current ‘dI’ flowing on this circumference is given as dR=2πrρ/ldr. Therefore,

$$d I = \frac{e}{d r} = - \frac{ϖ l {B}_{m} cos ϖ t}{2 ρ} r d r$$

Eddy current ‘I’ is represented by the following equation:

$$I = {\int }_{a}^{0} ⅆ l = - \frac{ϖ {a}^{2} l {B}_{m}}{4 ρ} cos ϖ t$$

As such, an effective value Iₑ of the current is shown as follows:

$${I}_{e} = \frac{ϖ {a}^{2} l {B}_{m}}{4 \sqrt{2 ρ}}$$

In the electric power ‘dp’ lost in the cylinder having a thickness ‘dr’, dp=(dl)²dR=(π/2ρ)ω²lB²cosωtr³dr. The lost electric power ‘P’ is given by the following equation:

$$p = {\int }_{0}^{a} ⅆ p = \frac{π}{8 ρ} {ϖ}^{2} {a}^{4} {B}_{m}^{2} {cos}^{2} ϖ t$$

An average e electric power ‘Pₘ’ versus a half-period may be represented by the following equation:

$${P}_{m} = \frac{ϖ}{π} {\int }_{0}^{\frac{π}{ϖ}} p ⅆ t = \frac{{ϖ}^{3} {a}^{2} {B}_{m}^{2} V}{16 πρ} {\int }_{0}^{\frac{π}{ϖ}} ( 1 + cos 2 ϖ t ) ⅆ t = \frac{{( π f a {B}_{m} )}^{2}}{4 ρ} V [ W ]$$

The average electric power Pₘ is the same as the eddy current loss Pₑ generated by the eddy current, and thus the eddy current loss per unit volume is as follows:

Pₑ∝σf²Bₘ²[W].

Wherein, σ [mho/m] is a conductance of iron core, and f[Hz] is a frequency, and Bₘ[wb/m²] is a maximal flux density.

Now, hysteresis core loss resulting from the magnetic field induced electrical circuit is described. When the current flows through the coil-wound magnetic circuit, an electromotive force corresponding to the electromotive force of a direct current circuit is generated, and a magnetic resistance corresponding to an electric resistance is formed in the magnetic material.

A magnetic field in the magnetic material having a length 1, a cross area S and a magnetic permeability μ is shown as Hₘ. A magnetic flux density in the magnetic material is Bₘ=μHₘ, so that a magnetic flux penetrating the sectional face S is represented by the following equation:

φ=BₘS=μHₘS[wb]

A magnetic potential difference between both ends of the magnetic material is given by

U=Hₘl

The magnetic potential difference U is divided by a magnetic flux Φ

Rₘ=U/φ=1/(μS) [AT/wb]

Wherein, Rₘ is a magnetic resistance and its unit is [AT/wb].

Hence, the magnetic resistance of the magnetic material is in proportion to the length l of wire and is in inverse proportion to the magnetic permeability μ times the cross area S. A reciprocal of the magnetic resistance Rₘ is called a permeance.

From the above equation, the following equation is obtained:

U=Rₘφ[AT]

This is referred to as Ohms' law in the magnetic circuit, and an energy density in the magnetic material is given as the following equation:

W=½HₘRₘ

Energy W accumulated in all of the magnetic material is obtained by an energy density w times the volume of the magnetic material. That is to say,

W=wlS=½HₘlBₘS

This equation defines hysteresis core loss.

Accordingly, it can be found that hysteresis core loss in the magnetic material is in proportion to the magnetic flux-permeating magnetic material volume.

As stated above, in the magnetic material, generation of heat by eddy current loss and hysteresis core loss results from the application of an external magnetic field, thereby the magnetic material itself producing heat.

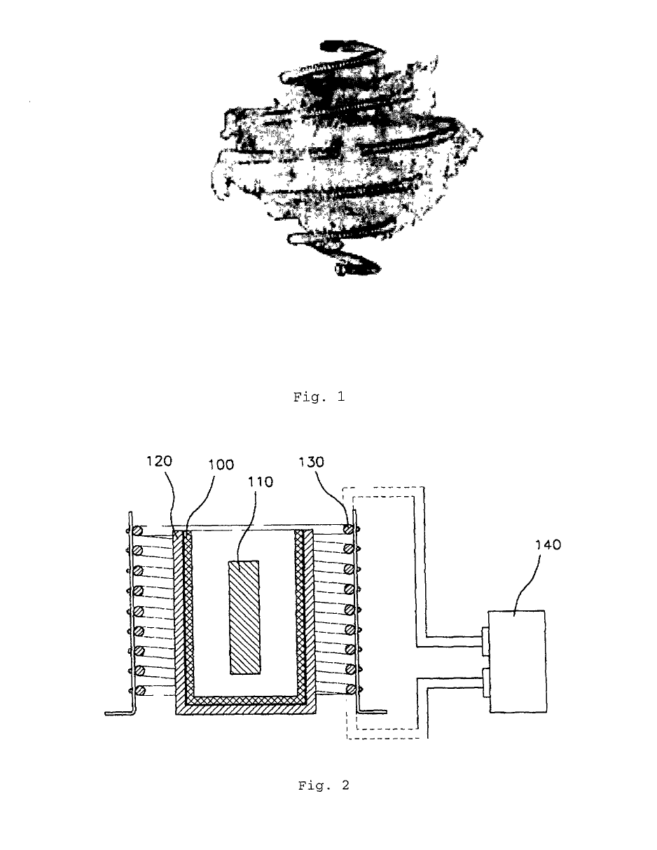

With reference to FIG. 1, there is shown a thermocoil in accordance with an embodiment of the present invention. As shown, the thermocoil assumes a spiral form and has such pili at its outer surface that it can block blood flow when being inserted into blood vessels. The thermocoil is fabricated to have flexibility by winding a wire of a predetermined length to a coil form as well as to a spiral form.

The thermocoil is made of a wire material which is excellent in terms of corrosion resistance and biocompatibility, such as duplex stainless steel, nickel-copper alloy, iron-nickel alloy, palladium-cobalt alloy, and palladium-nickel alloy. For heat induction, the material is annealed at 200-1,500° C. to have α phase and γ phase or martensitic phase. In the case of a duplex stainless steel wire, α phase and martensitic phase show a magnetic characteristic while γ phase is non-magnetic.

The domain portion between the magnetic phase and non-magnetic phase can be controlled by the heat treatment process. That is, heat treatment gives rise to a change in the magnetic and non-magnetic phase domains of magnetic materials.

Exhibiting magnetic characteristics, α phase and martensitic phase domains emit a large quantity of heat by eddy current loss and hysteresis core loss when they are placed in external magnetic fields. As for a γ phase domain, on the other hand, it is of non-magnetic characteristic and has heat created only by eddy current loss so that the heat value becomes lower.

In the thermocoil made of duplex stainless steel wire according to the present invention, the heat value can be controlled by regulating the domain portion exerting the magnetic characteristic and the non-magnetic characteristic through the heat treatment process.

The thermocoil made of iron-nickel alloy shows magnetic permeability which varies with the nickel content or heat treatment. Thus, the thermocoil, when subjected to an external magnetic field, can emit heat in a controllable quantity depending on the strength of such a magnetic field.

There is a thermoguide wire(not shown) in accordance with another embodiment of the present invention. The thermo-guide wire assumes a coil form which allows catheters to be inserted into lumens with ease. The thermocoil is fabricated to have flexibility by winding a wire of a predetermined length to a coil form.

For use in the thermoguide wire, the wire material is required to be excellent in terms of corrosion resistance and biocompatibility. Those materials that meet these requirements are exemplified by duplex stainless steel, nickel-copper alloy, iron-nickel alloy, palladium-cobalt alloy, and palladium-nickel alloy. For heat induction, the material is annealed at 200-1,500° C. to have α phase and γ phase or martensitic phase. In the case of a duplex stainless steel wire, α phase and martensitic phase show a magnetic characteristic while γ phase is non-magnetic.

Through the heat treatment, the domain portion between the magnetic phase and non-magnetic phase can be adjusted. That is, heat treatment brings about a change in the magnetic and non-magnetic phase domains of magnetic materials.

With magnetic characteristics, α phase and martensitic phase domains emit a large quantity of heat by eddy current loss and hysteresis core loss when they undergo influence of external magnetic fields. As for a γ phase domain, on the other hand, it is of non-magnetic characteristic and has heat created only by eddy current loss so that the heat value becomes lower.

By regulating the domain portion exerting the magnetic characteristic and the non-magnetic characteristic through the heat treatment process, the heat value can be controlled in the thermo-guide wire made of duplex stainless steel wire according to the present invention.

Also, the thermoguide wire made of iron-nickel alloy shows magnetic permeability depending on the nickel content or heat treatment. Thus, the thermoguide wire, when subjected to an external magnetic field, can emit heat in a controllable quantity according to the strength of such a magnetic field.

There is a thermostent (not shown) according to a further embodiment of the present invention. The thermostent is inserted to lumens such as a coronary artery to support passageways of the lumen and to prevent restenosis or expansion of the lumen, and comprises a mesh type hollow tube arranged in a zigzag configuration. The mesh tubular stent is made of a wire or tubular material with excellent corrosion resistance and biocompatibility. Examples of the material useful in the present invention include duplex stainless steel, nickel-copper alloy, iron-nickel alloy, palladium-cobalt alloy, and palladium-nickel alloy, which is annealed at 200-1,500° C. to have α phase and γ phase or martensitic phase.

The mesh type tubular stent can be fabricated by weaving wires of a predetermined length crossways, like knitting with a warp and a weft, to make a hollow cylindrical stent body having a net structure with a plurality of diamond-shaped meshes, or by cutting a tube to a predetermined length and processing the tube to a mesh form.

In the case of the duplex stainless steel wire, α phase and martensitic phase show a magnetic characteristic while γ phase is non-magnetic.

The domain portion between the magnetic phase and non-magnetic phase can be controlled by the heat treatment process. That is, when undergoing the heat treatment process, magnetic materials have the magnetic and non-magnetic phase domains changed.

Exhibiting magnetic characteristics, α phase and martensitic phase domains emit a large quantity of heat by eddy current loss and hysteresis core loss in the presence of an external magnetic field. As for a γ phase domain, on the other hand, it is of non-magnetic characteristic and has heat created only by eddy current loss so that the heat value becomes lower.

By regulating the domain portion exerting the magnetic characteristic and the non-magnetic characteristic through the heat treatment process, the heat value can be controlled in the thermostent made of duplex stainless steel wire according to the present invention.

The thermostent made of copper-nickel alloy shows magnetic permeability which varies with the nickel content or heat treatment. Thus, the thermostent, when subjected to an external magnetic field, can emit heat in a controllable manner depending on the strength of such a magnetic field.

Below, a description will be given of the thermo implants, that is, thermocoil, thermoguide wire, and thermostent, made of duplex stainless steel wire.

At lower than their magnetic transition temperature, the thermo implants made of duplex stainless steel wire come to be composed of the magnetic phase, i.e., α phase and martensitic phase, thus showing such high magnetic permeability as to emit heat in a large quantity in the presence of an external magnetic field. Above magnetic transition temperature, on the other hand, only non-magnetic γ phase c is present in the thermo implants which are thus not heated any further, but cooled even in the presence of an external magnetic field. As cooling is progressed, the implants undergo phase transition to regain the lost magnetism, that is, the magnetic characteristic, and thus restore the magnetic permeability. Then, the thermo implants are again heated so the temperature is increased. This phase transition mechanism is repeated with maintenance of the implants at constant temperatures.

Having generally described this invention, a further understanding can be obtained by reference to certain specific examples which are provided herein for purposes of illustration only and are not intended to be limiting unless otherwise specified.

### EXAMPLE 1

First, an examination was made of the heating properties of coils.

Generally, coils are prepared from wires with predetermined lengths and diameters. In the present invention, a coil is made of the duplex stainless steel wire which is thermally treated as described above. A wire with a predetermined length and diameter was wound to a coil form and then to a spiral form to give a spiral thermocoil.

The coil was measured for heating properties and heating rate.

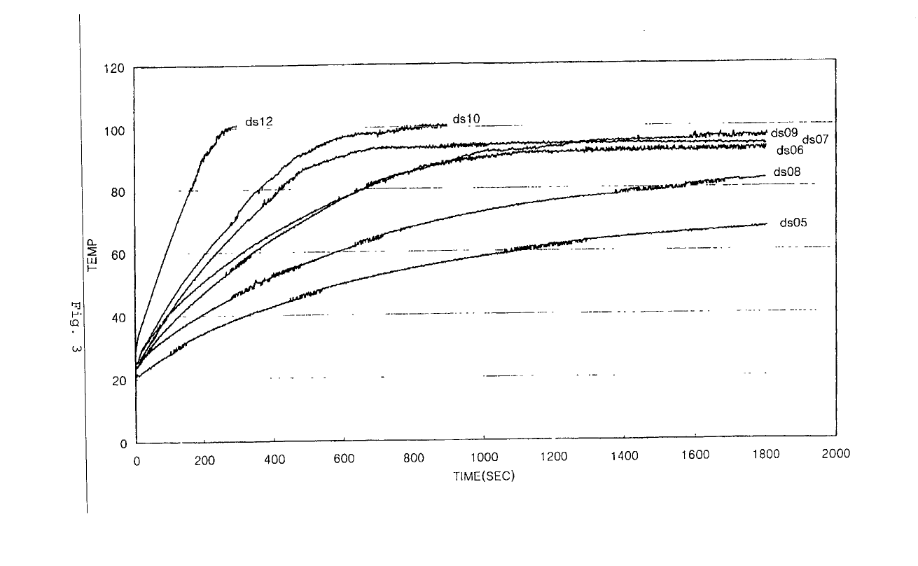

Referring to FIG. 2, there is shown a heat value-measuring apparatus. The apparatus, as shown, comprise a chamber 100 in which a coil 110 is positioned vertically and distilled water is filled. The chamber 100 is surrounded by an adiabatic material 120. A magnetic field generator 130 surrounds the chamber 100 at a certain distance apart therefrom. Supplied with power from a power supply 140, the magnetic field generator 140 applies a magnetic field to the coil 110.

By operation of the magnetic field generator 130, the coil 110 was induced to generate heat. Using thermocouples, the temperature data was gathered at four points and averaged.

The heating properties and heating rate depending on coil diameter are summarized in Table 1, below. FIG. 3 is a graph in which the temperature properties of the coil are plotted versus time.

TABLE 1

| | Dimension | | | | Heating |
|---|---|---|---|---|---|
| | Diameter | Height | Weight | Max. Temp | Rate |
| Specimen | (mm) | (mm) | (g) | () | () |
| ds05 | 0.49 | 24.9 | 0.04 | 67.4 | 2 |
| Ds06 | 0.59 | 24.5 | 0.05 | 93.3 | 4.7 |
| Ds07 | 0.69 | 25.0 | 0.07 | 95.2 | 7.7 |
| Ds08 | 0.79 | 24.7 | 0.09 | 83 | 2.1 |
| Ds09 | 0.89 | 25.5 | 0.12 | 97.9 | 4 |
| Ds10 | 0.99 | 24.4 | 0.15 | 100.8 | 8.3 |
| Ds12 | 1.19 | 25.8 | 0.19 | 101.1 | 17.6 |

As seen in Table 1 and FIG. 3, the highest temperature the coli of the present invention can reach is increased with increasing of the diameter of the coil.

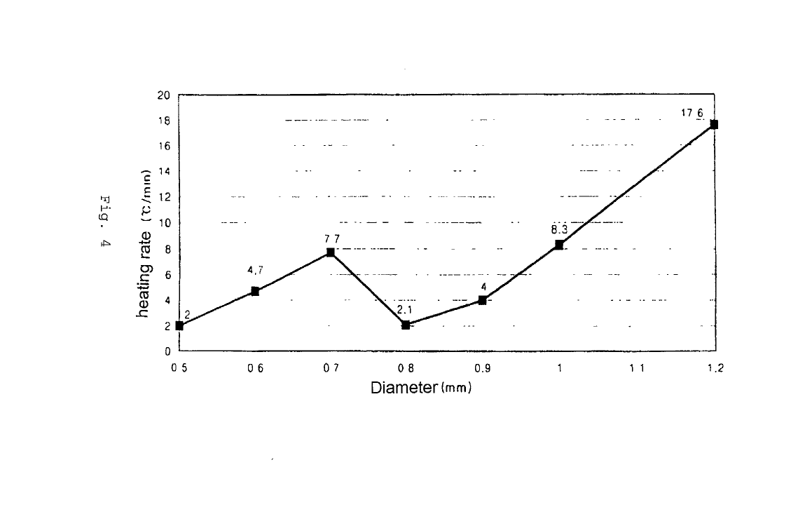

FIG. 4 is a curve in which the heating rate of the coil is plotted versus the diameter. This curve also shows that the larger the diameter is, the higher the heating rate is.

This phenomenon is caused because hysteresis core loss is in proportion to a cross sectional area of the wire.

As described above, the coil manufactured from the wire which underwent the thermal treatment process shows the highest heat value in response to an external magnetic field, depending on the diameter thereof. In other words, desired highest heat values can be determined by regulating the diameter of the coil. Therefore, a site to be treated can be maintained at a desired temperature by use of such a coil. The thermocoil is fabricated by further winding the coil to a spiral form and bonding pili thereonto. When the thermocoil is directly inserted into, for example, blood vessels, the pili act to close the vessels.

The heating properties of the spiral thermocoil were found to be similar to those of the simple coil. This is believed to be attributed to the fact that the magnetic flux penetrating the cross sectional area of the simple coil is similar to that penetrating the cross sectional area of the spiral coil.

Operational effects of such a structure are as follows.

A duplex stainless steel wire is cut into pieces of a predetermined length. When the cut wire is annealed at 200-1500° C., a domain portion between the magnetic α phase and martensitic phase and the non-magnetic γ phase is changed to bring about alterations in magnetic permeability and magnetic transition temperature. When being subjected to an external magnetic field, the duplex stainless steel wire can maintain its temperature at 30-200° C. By thermally treating the duplex stainless wire, the heating temperature can be controlled.

The cut wire is wound to a coil form which is then further wound to a spiral form, followed by bonding pili onto the resulting spiral coil to obtain the thermocoil of the present invention.

The spiral coil was prepared the wires which had been cut and subjected to heat treatment. However, the same results could be obtained from the spiral coil which was thermally treated after being fabricated with wires which were not thermally previously.

Into blood vessels around a site to be treated, the thermocoil is inserted. Once the thermocoil is inserted, the blood flow in the vessels is interrupted by the structure and pili of the thermocoil. When an external magnetic field is applied around the thermocoil inserted blood vessel with variation in magnetic filed intensity, the thermocoil generates heat by itself and reaches a predetermined temperature to induce hyperthermia whereby cancerous tissues can be necrotized or physiological changes are caused to increase the therapeutic effect versus diseases.

### EXAMPLE 2

In this example, guide wires were examined for heating properties.

Before preparation of the guide wires, heating properties of duplex stainless steel wire were examined for dependence on heat treatment temperature and diameter.

FIG. 2 is a schematic diagram showing a heat value-measuring apparatus. The apparatus, as shown, comprise a chamber 100 in which a coil 110 is positioned vertically and distilled water is filled. The chamber 100 is surrounded by an adiabatic material 120. A magnetic field generator 130 surrounds the chamber 100 at a certain distance apart therefrom. Supplied with power from a power supply 140, the magnetic field generator 140 applies a magnetic field to the coil 110.

By operation of the magnetic field generator 130, the coil 110 generates heat by itself. The temperature was averaged from temperature data of four points using a thermocouple.

First, the heating properties varying with the heat treatment temperature were investigated.

FIG. 6 shows heat value per unit weight and unit time according to the heat treatment temperature of the duplex stainless steel wire with a diameter of 0.16 mm. The heating properties were measured in wires which were thermally treated at 300, 500, 700, 800, 900, 1100 and 1300° C., or not treated. From the results of this figure, it can be seen that the stainless steel wire which was not thermally treated has a maximal heat value per unit time and unit weight, and the heat value is decreased with increasing of the heat treatment temperature.

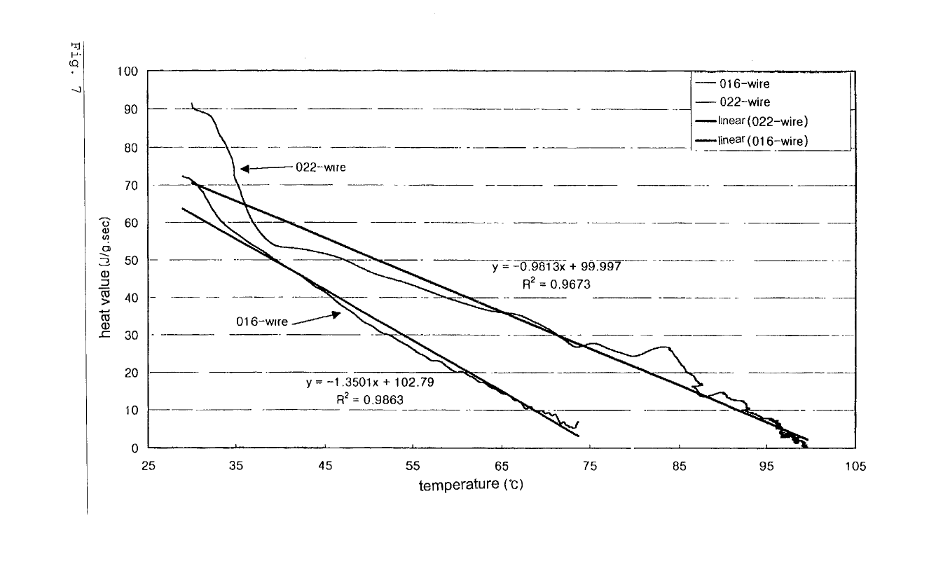

It is apparent from FIG. 7 that the heat values of the thermally treated duplex stainless steel wires used in the present invention decrease with increasing of the temperature of the wires. It is believed that the heat value is decreased as the temperature of the stainless steel wire approaches the magnetic transition temperature.

Additionally, the larger the diameter of the wire is, the higher the heat value at a specific temperature is. As stated above, this phenomenon is attributed to the fact that hysteresis core loss is in proportion to a cross sectional area of the wire.

The guide wire was fabricated by winding the duplex stainless steel wire with a predetermined length and diameter to a coil form. Before manufacture of the thermoguide wire, the duplex stainless steel material was subjected to heat treatment.

An examination of the guide wire for heating properties and heating rate showed that the highest temperature the coli of the present invention can reach is increased with increasing of the diameter of the coil. The heating rate was also found to be increased as the diameter of the wire is increased. This phenomenon can be explained by the fact that hysteresis core loss is in proportion to a cross sectional area of the wire.

As described above, the guide wire manufactured from the wire which underwent the thermal treatment process shows the highest heat value in response to an external magnetic field, depending on the diameter thereof. In other words, desired highest heat values can be determined by regulating the diameter of the coil. Therefore, a site to be treated can be maintained at a desired temperature by use of such a guide wire.

### EXAMPLE 3

In this example, the guide wires prepared above were applied to animals. For use in hyperthermia, the guide wires were examined for heating properties in pig liver.

Animal experiments were carried out by use of the apparatus illustrated in FIG. 2. In the chamber 100 of the apparatus, as shown in FIG. 2, the pig liver is positioned, followed by filling distilled water in the chamber 100.

Prior to positioning of the pig liver, a space corresponding to a size of the guide wire is formed in the pig liver to mount the guide wire therein. The pig liver having the inserted guide wire is positioned in such a way that the guide wire is vertically oriented. The chamber 100 is surrounded by an adiabatic material 120. A magnetic field generator 130 surrounds the chamber 100 at a certain distance apart therefrom. Supplied with power from a power supply 140, the magnetic field generator 140 applies a magnetic field to the pig liver.

By operation of the magnetic field generator 130, the guide wire was induced to generate heat. Using thermocouples, the temperature was measured at seven points 0, 3, 6, 9, 12, 15 and 18 mm from the center of the guide wire.

Used in this example was a guide wire of a dimension which is most widely used at present: it was 0.87 in diameter and 46 cm in length.

Heating experiments were conducted with a thermoguide wire encapsulated with a tube and a naked thermo-guide. The temperature measurement results are given in Table 2, below.

TABLE 2

| | Temperature Difference (° C.) | | | | | | |
|---|---|---|---|---|---|---|---|
| | CH 1 | CH 2 | CH 3 | CH 4 | CH 5 | CH 6 | CH 7 |
| Capsulated | 28.8 | 19.1 | 16 | 9.8 | 8 | 7.1 | 4.9 |
| Naked | 28.6 | 14 | 14 | 10.2 | 8.8 | 7.6 | 4.7 |

Note:
temperature difference = final temperature-initial temperature,
channel 1 showing the result for center temperature,
channels 2 to 7 showing the results for temperature measurement at points 3, 6, 9, 12, 15 and 18 mm from the center.

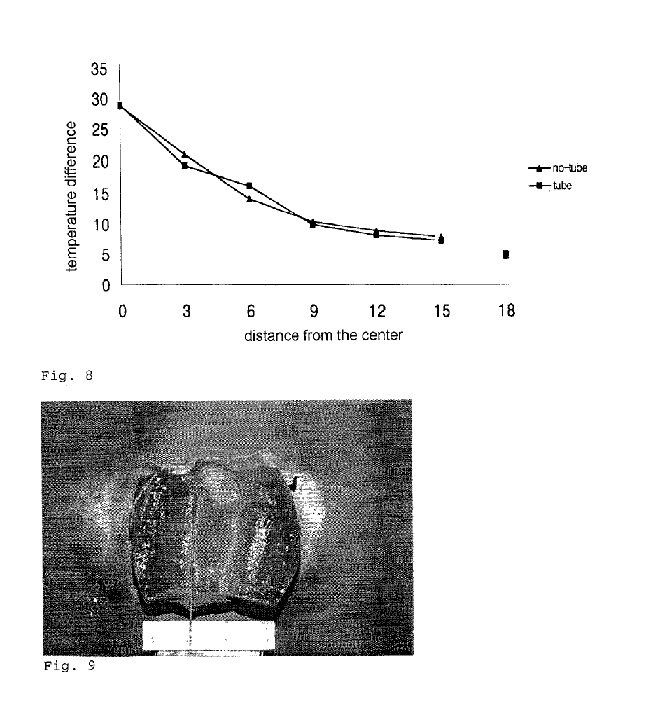

FIG. 8 is a graph in which the temperature difference versus the distance from the center for temperature measurement is plotted for stents.

No great differences in heat value were not observed between the naked thermoguide wire and the encapsulated thermoguide wire, as shown in Table 2 and FIG. 8. Also, it was observed that the guide wires generated more heat at their central portions than at their end portions. Therefore, the central portion is suitable for use in hyperthemic therapy of diseases.

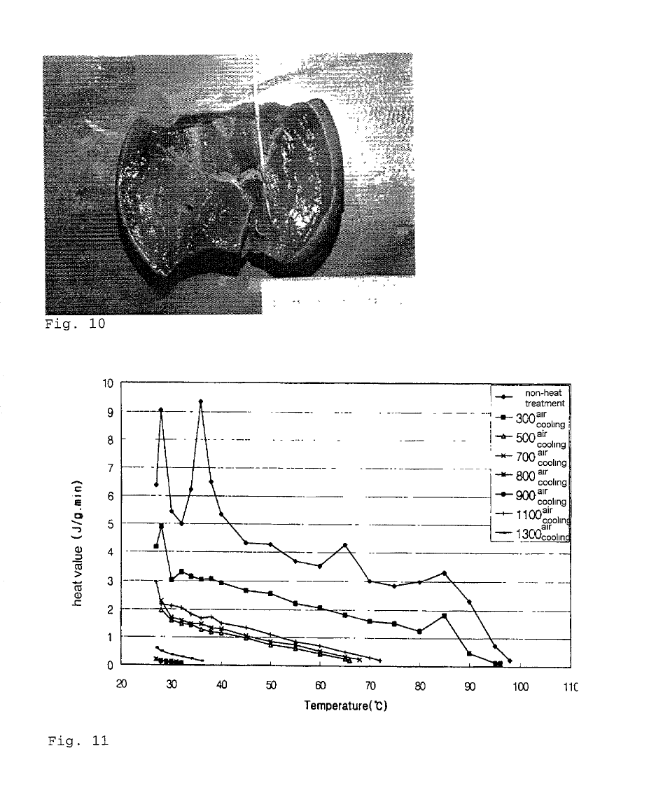

As shown in FIG. 9 or 10, protein denaturation of the pig liver is caused by the guide wire according to the present invention. From the drawings, it can be seen that the denaturation occurs to lesser extents at points more distant from the stent-inserted portion. Also, it is found that the denaturation by the central portion of the guide wire is greater than that by the end portion.

As mentioned above, the controllable parameters affecting the heat that the thermo-guide wire generates by itself according to the present invention include the heat treatment temperature and wire diameter.

Operational effects of such a guide wire structure are as follows.

A duplex stainless steel wire is cut into pieces of a predetermined length. When the cut wire is annealed at 200-1500° C., a domain portion between the magnetic α phase and martensitic phase and the non-magnetic γ phase is changed to bring about alterations in magnetic permeability and magnetic transition temperature. When being subjected to an external magnetic field, the duplex stainless steel wire can maintain its temperature at 30-200° C. By thermally treating the duplex stainless wire, the heating temperature can be controlled.

The cut wire was wound to a coil form to give a thermoguide wire.

The guide wire was prepared from the wires which had been cut and subjected to heat treatment. However, the same results could be obtained from the stent which was thermally treated after being fabricated with wires which were not thermally previously.

After the guide wire is inserted into a lumen to be treated, a catheter is easily introduced into the lumen through the guide wire.

When the external magnetic field is applied around the guide wire-inserted lumen, the guide wire generates inductive heat in response to a change in the intensity of the magnetic field and thus reaches a predetermined temperature to perform hyperthermia whereby cancerous tissues can be necrotized or physiological changes are caused to increase the therapeutic effect versus diseases.

### EXAMPLE 4

In this example, an examination was made of the heating properties of a thermostent, which vary with its design, wire diameter and heat treatment temperature.

Before preparation of the stent, heating properties of duplex stainless steel wire were examined for dependency on heat treatment temperature and diameter.

With reference to FIG. 11, there are plotted heat values per unit weight and unit time versus the heat treatment temperature of the duplex stainless steel wire with a diameter of 0.16 mm. The heating properties were measured in wires which were thermally treated at 300, 500, 700, 800, 900, 1100 and 1300° C., or not treated. As apparent from the data of this figure, the stainless steel wire which was not thermally treated has the highest heat value per unit time and unit weight, and the heat value is decreased with increasing of the heat treatment temperature.

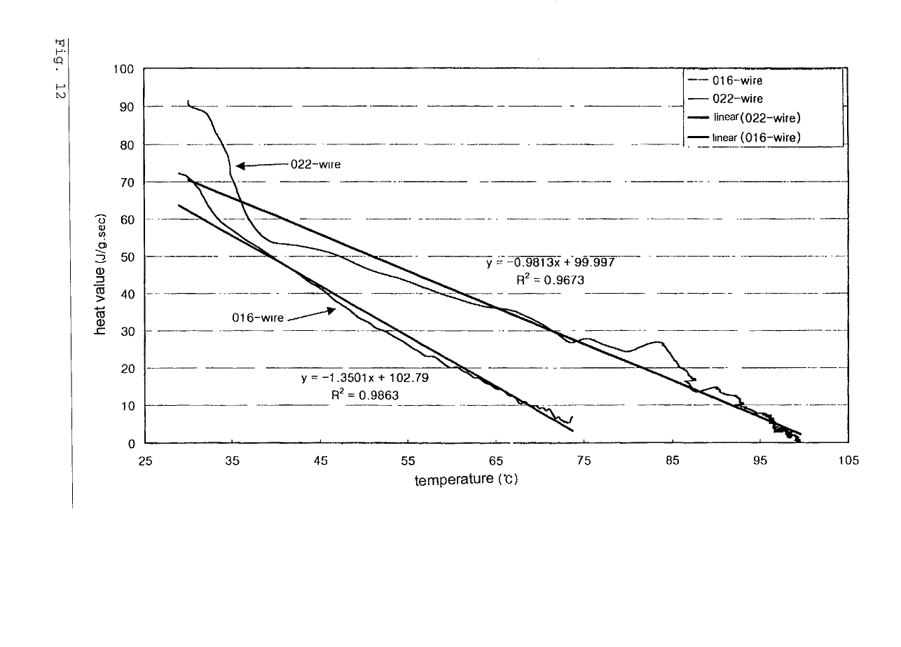

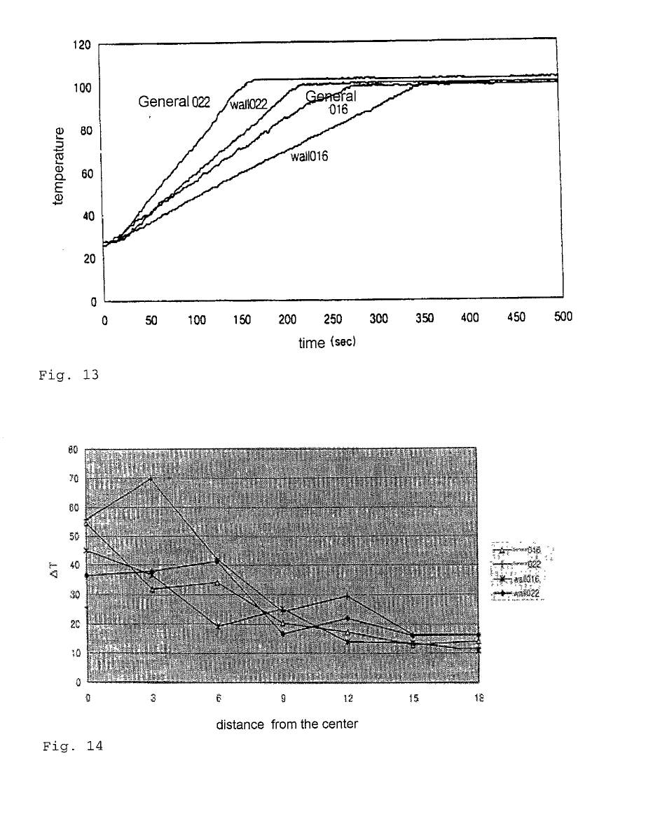

Also, it is apparent from FIG. 12 that the heat values of the thermally treated duplex stainless steel wires used in the present invention decrease with increasing of the temperature of the wires. It is believed that the heat value is decreased as the temperature of the stainless steel wire approaches the magnetic transition temperature.

Additionally, the larger the diameter of the wire is, the higher the heat value at a specific temperature is. As stated above, this phenomenon is attributed to the fact that hysteresis core loss is in proportion to a cross sectional area of the wire.

The thermostent was manufactured from the duplex stainless steel wire which shows such properties. Before manufacture of the thermostent, the duplex stainless steel material was subjected to heat treatment. Used were wires that were 0.16 mm and 0.22 mm in diameter. Two types of thermostents were manufactured: a general type is made of a plurality of wires, each winding in 11/7 turns from a starting portion to an ending portion; and a wall type thermostent has a structure in which wires are longitudinally and transversely crossed, so as not to reduce the outer diameter.

The dimensions and characteristics of the prepared thermostent stents are shown in Table 3, below.

TABLE 3

| | Wire | Inner | Outer | Stent | | Sur- |
|---|---|---|---|---|---|---|
| | Length | Diameter | Diameter | length | Weight | face |
| Stent | (mm) | (mm) | (mm) | (mm) | (g) | area |
| General 016 | 95 | 6 | 6.24 | 52.06 | 0.1483 | 47.79 |
| General 022 | 92 | 6 | 6.62 | 51.09 | 0.2673 | 60.76 |
| Wall 016 | 116 | 6 | 6.64 | 48.22 | 0.1857 | 58.35 |
| Wall 022 | 119 | 6.1 | 7.03 | 48.26 | 0.3488 | 78.58 |

The thermostents were tested for heating properties by use of the device shown in FIG. 2. The results are summarized in Table 4, below.

TABLE 4

| | | | | 90% | | | | | | |
|---|---|---|---|---|---|---|---|---|---|---|
| | | Max. | | Temp. | | | Heat | Heat | Heat | Heat |
| | Init. | heat | 90% | reach | Heat | Heat | Value/ | Value/ | Value/ | Value/ |
| | Temp. | Temp. | Temp. | time | Rate | Value | time | wt | Area | Time × Area |
| Stent | (° C.) | (° C.) | (° C.) | (sec) | (° C./sec) | (J) | (J/sec) | (J/g) | (J/mm²) | (J/mm²sec) |
| General | 25.5 | 100 | 67.05 | 141 | 0.475 | 2494.56 | 17.69 | 119.30 | 5.220 | 0.037 |
| 016 | | | | | | | | | | |
| General | 27.3 | 103.3 | 68.40 | 93 | 0.735 | 2477.82 | 24.64 | 99.68 | 4.078 | 0.044 |
| 022 | | | | | | | | | | |
| Wall 016 | 27.5 | 101.1 | 66.24 | 185 | 0.358 | 2464.42 | 13.32 | 71.71 | 4.224 | 0.023 |
| Wall 022 | 27.8 | 101.0 | 65.88 | 121 | 0.544 | 2451.03 | 20.25 | 58.07 | 3.119 | 0.026 |

Note:
90% temperature = maximal heating temperature-(maximal heating temperature-initial temperature) × 0.1.
heat rate = gradient of from initial temperature to 90% temperature.
heat value = 9R × (maximal heating temperature-initial temperature) × water amount, wherein R is 8.31451 J/mol · K.

With reference to FIG. 13, there are illustrated heating properties of the stent. At the same diameter, as can be seen in Table 4 and FIG. 13, the general type stent generates more heat than does the wall type stent. Based on the fact that hysteresis core loss is in proportion to the cross sectional area and length of the wire through which the magnetic field passes, the generation of more heat in the general type stent is attributed to the wire structure of the general type stent, in which wires are oriented in substantially perpendicular directions, thereby allowing a magnetic field to pass through a greater area and length.

In addition, the larger the diameter of the wire, the more the generated heat.

As apparent from the data of Table 4, the stent having large diameter has higher heat value per unit time, but lower heat value per unit weight than the stent having small diameter, regardless of types of the stent.

From the results, it can be confirmed that the stent made of thicker wire produces more heat, so that a desired heat value and temperature increase can be obtained by regulating the diameter of the wire.

### EXAMPLE 5

In this example, the stents prepared above were applied to animals. For use in hyperthermia, the stents were examined for heating properties in pig liver.

Animal experiments were carried out by use of the apparatus illustrated in FIG. 2. In a chamber 100 of the apparatus, as shown in FIG. 2, the pig liver is positioned, followed by filling distilled water in the chamber 100.

Prior to positioning of the pig liver, a space corresponding to a size of the stent is formed in the pig liver to mount the stent therein. The pig liver having the inserted stent is positioned in such a way that the stent is vertically oriented. The chamber 100 is surrounded by an adiabatic material 120. A magnetic field generator 130 surrounds the chamber 100 at a certain distance apart therefrom. Supplied with power from a power supply 140, the magnetic field generator 140 applies a magnetic field to the pig liver.

By operation of the magnetic field generator 130, the stent was induced to generate heat. Using thermocouples, the temperature was measured at seven points apart from the center of the stent by 0, 3, 6, 9, 12, 15 and 18 mm.

The characteristics of the used stents were the same as that of Table 1 of Example 1.

The temperature measurement results are given in Table 5, below.

TABLE 5

| | Temperature Difference (° C.) | | | | | | |
|---|---|---|---|---|---|---|---|
| Stent | CH 1 | CH 2 | CH 3 | CH 4 | CH 5 | CH 6 | CH 7 |
| General | 54.6 | 31.8 | 34.1 | 20.4 | 17.3 | 12.9 | 14.1 |
| 016 | | | | | | | |
| General | 55.7 | 69.6 | 42.0 | 24.2 | 29.5 | 16.4 | 16.4 |
| 022 | | | | | | | |
| Wall 016 | 44.9 | 36.6 | 19.2 | 25.2 | 13.9 | 13.5 | 10.8 |
| Wall 022 | 36.4 | 37.8 | 41.2 | 16.6 | 22.0 | 16.1 | 16.2 |

Note:
temperature difference = final temperature-initial temperature,
channel 1 showing the result for center temperature,
channels 2 to 7 showing the results for temperature measurement at points 3, 6, 9, 12, 15 and 18 mm from the center.

With reference to FIG. 8, the temperature difference versus the distance from the center for temperature measurement is plotted for stents, showing that general type stents are larger in the temperature difference than are wall type stents, that is, indicating that the heating temperature of the general type stent is higher than that of the wall type stent. This is because each wire in the general type stent is oriented in two directions that are substantially perpendicular to each other. Meanwhile, the general type stents, although lighter, have higher heat value than the wall type stents. The reason is that each wire in the general type stents is oriented in more perpendicular directions, compared to the wall type stents. By controlling the intersection angles of wires, the heat value of the stent can be regulated.

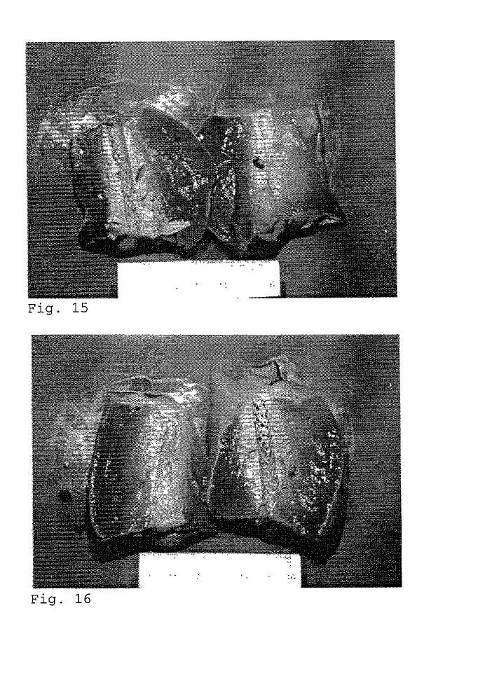

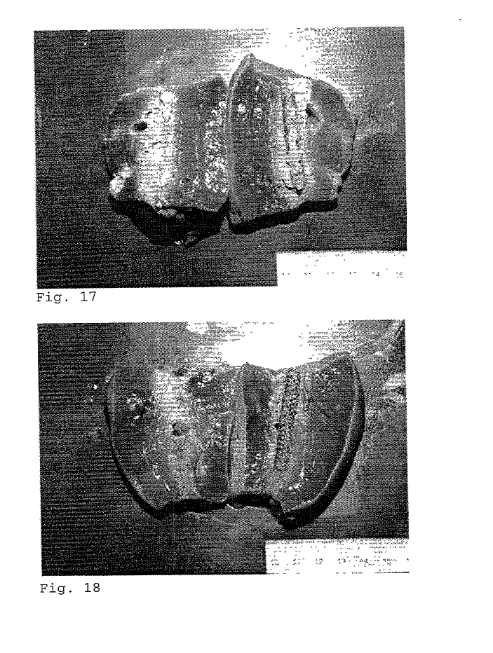

As shown in FIGS. 15 to 17, protein denaturation of the pig liver is caused by the stent according to the present invention. From the drawings, it can be seen that the denaturation occurs to lesser extents at points more distant from the stent-inserted portion.

As mentioned above, the controllable parameters affecting the heat that the thermostent generates by itself according to the present invention include the heat treatment temperature, wire diameter and direction of magnetic field-generator.

In accordance with the present invention, the heat-treated duplex stainless steel stent is combined with a cylindrical form of shape memory alloy. In this regard, the heat-treated duplex stainless steel stent is wound to peripheral surface of a cylindrical shape memory alloy, so that the resulting structure has the structural characteristic of retaining its cylindrical form at a specific temperature and the thermal characteristic of generating inductive heat in the presence of an external magnetic field. If an inductive heat is generated from the duplex stainless steel stent by the application of the external magnetic field to heat the complex structure to a specific temperature, the shape memory alloy is expanded in a tubular form by the heat delivered thereto, thereby maintaining the entire stent form. Also, the inductive heat generated from the duplex stainless steel stent is delivered outside.

Operational effects of such a complex structure are as follows.

A duplex stainless steel wire is cut into pieces of a predetermined length. When the cut wire is annealed at 200-1500° C., a domain portion between the magnetic α phase and martensitic phase and the non-magnetic γ phase is changed to bring about alterations in magnetic permeability and magnetic transition temperature. When being subjected to an external magnetic field, the duplex stainless steel wire can maintain its temperature at 30-200° C. By thermally treating the duplex stainless wire, the heating temperature can be controlled.

The general type stent and the wall type stent were manufactured by weaving the cut wires crossways, like knitting with a warp and a weft, to make a hollow cylindrical stent body having a net structure with a plurality of diamond-shaped meshes.

The stent was prepared from the wires which had been cut and subjected to heat treatment. However, the same results could be obtained from the stent which was thermally treated after being fabricated with wires which were not thermally previously.

The completed stent is inserted into the body lumen to be treated, whereby the stent maintains the intraluminal tubular structure. When the external magnetic field is applied around the stent-inserted lumen, the stent generates the heat by such application of the external magnetic field and thus reaches a predetermined temperature. Therefore, restenosis of the lumen is prevented and necrosis of tumor tissue is caused, or physiological function of the lumen tissue is altered, thus the therapeutic effect versus the diseases being increased.

Now, a point of view is turned to the thermocoil, thermoguide wire, and thermostent made of iron-nickel alloy.

Like duplex stainless steel, iron-nickel alloy is subjected to the same heat treatment, and the same type implants are manufactured.

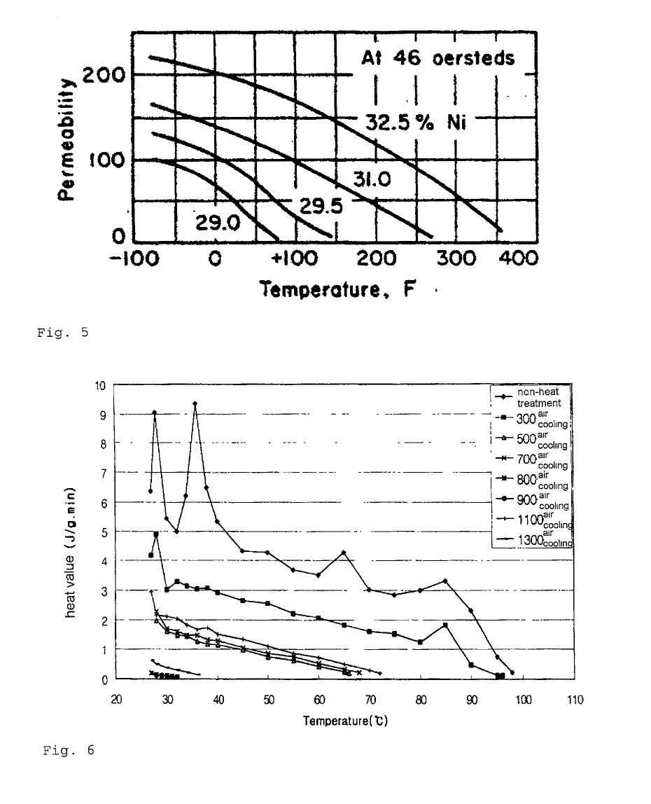

As illustrated in FIG. 5, the iron-nickel alloy subjected to heat treatment has higher magnetic permeability as nickel content is increased. That is, high nickel content results in relatively larger heat value.

In addition, when the implant reaches a predetermined temperature, magnetic permeability is drastically decreased. In other words, the iron-nickel alloy generates heat under external magnetic field until reaching magnetic transition temperature, whereas the heat value is drastically decreased after reaching magnetic transition temperature.

The thermostent, thermoguide wire, thermocoil made of iron-nickel alloy have similar properties to those of the corresponding implants made of duplex stainless steel wire.

As described above, the thermo implants of the present invention are useful in hyperthermia.

In addition to maintaining intraluminal passageways open and preventing the intraluminal growth of tissues and the restenosis or expansion of the lumens, the thermostent generates heat by itself in the body in response to the application of an external magnetic field without a separate electrical connection, thereby inducing necrosis or physiological changes at the target site and neighboring tissues to improve therapeutic effects at the target site.

The thermocoil of the present invention can be inserted into blood vessels to interrupt the blood flow and can generate heat by itself in response to the application of an external magnetic field without a separate electrical connection to the exterior, to maintain the target site at a predetermined temperature, whereby the target site and neighboring tissues are caused to undergo necrosis or physiological changes to improve therapeutic effects on the target site.

The thermoguide wire of the present invention can be inserted into lumens of the body to facilitate the safe and easy insertion of catheters and generate heat by itself in response to the application of an external magnetic field without a separate electrical connection to the exterior, so as to maintain the target site at a predetermined temperature, thereby inducing necrosis or physiological changes at the target site and neighboring tissues to improve therapeutic effects at the target site.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A thermostent for insertion into the lumen, having a mesh tubular form made of a heat-treated, magnetic material, which can generate heat by itself in response to the application of an external magnetic field thereto.

2. The thermostent as set forth in claim 1, wherein the material is selected from the group consisting of duplex stainless steel, nickel-copper alloy, iron-nickel alloy, palladium-cobalt alloy, and palladium-nickel alloy.

3. The thermostent as set forth in claim 1, wherein the material is thermally treated at 200-1,500° C.

4. The thermostent as set forth in claim 1, wherein the stent has a maximal heating temperature of 30-200° C.

5. The thermostent as set forth in claim 1, wherein the heat-treated magnetic material is wound on a peripheral surface of a shape memory alloy in a mesh form.

6. A thermocoil for insertion into the lumen, having a spiral form made of thermally treated, magnetic wire material, which functions to generate heat by itself in response to the application of an external magnetic field thereto and to block blood flow when being inserted into blood vessels.

7. The thermocoil as set forth in claim 6, wherein the material is selected from the group consisting of duplex stainless steel, nickel-copper alloy, iron-nickel alloy, palladium-cobalt alloy, and palladium-nickel alloy.

8. The thermocoil as set forth in claim 6, wherein the material is thermally treated at 200-1,500° C.

9. The thermocoil as set forth in claim 6, wherein the thermo-coil has a maximal heating temperature of 30-200° C.

10. The thermocoil as set forth in claim 6, wherein the heat-treated magnetic material has pili bonded thereto.

11. A thermoguide wire for insertion into the lumen, having a coil form made of thermally treated, magnetic wire material, which generates heat by itself in response to the application of an external magnetic field thereto.

12. The thermoguide wire as set forth in claim 11, wherein the material is selected from the group consisting of duplex stainless steel, nickel-copper alloy, iron-nickel alloy, palladium-cobalt alloy, and palladium-nickel alloy.

13. The thermoguide wire as set forth in claim 11, wherein the material is thermally treated at 200-1,500° C.

14. The thermoguide wire as set forth in claim 11, wherein the thermo-guide wire has a maximal heating temperature of 30-200° C.
